SEQUENCE LISTING (17 sequences)

SEQ ID NO: 1 - protein, 302 aa, Homo sapiens
MHCGPPDMVCETKIVAAEDHEALPGAKKDALLAAAGAMWPPLPAAPGPAAAPPAPPPAPVAQPHGGAGGAGPPGGRGVCIREFRAAEQEAARRIFYDGIMERIPNTAFRGLRQHPRAQLLYALLAALCFAVSRSLLLTCLVPAALLGLRYYYSRKVIRAYLECALHTDMADIEQYYMKPPGSCFWVAVLDGNVVGIVAARAHEEDNTVELLRMSVDSRFRGKGIAKALGRKVLEFAVVHNYSAVVLGTTAVKVAAHKLYESLGFRHMGASDHYVLPGMTLSLAERLFFQVRYHRYRLQLREE

SEQ ID NO: 2 - protein, 6 aa, Artificial Sequence
  Description of Artificial Sequence Synthetic 6xHis tag
HHHHHH

SEQ ID NO: 3 - protein, 4 aa, Artificial Sequence
  Description of Artificial Sequence Synthetic peptide
KEQN

SEQ ID NO: 4 - protein, 14 aa, Homo sapiens
IVAAEDHEALPGAK

SEQ ID NO: 5 - protein, 6 aa, Homo sapiens
LFFQVR

SEQ ID NO: 6 - protein, 299 aa, Mus musculus
MHCGPPDMVCETKIVATEDHEALPGAKKDALLVAAGAMWPPLPAAPGPAAAPPPAAGPQPHGGTGGAGPPEGRGVCIREFRAAEQEAARRIFYDGILERIPNTAFRGLRQHPRTQLLYALLAALCFAVTRSLLLTCLVPAGLLALRYYYSRKVILAYLECALHTDMADIEQYYMKPPGSCFWVAVLDGNVVGIVAARAHEEDNTVELLRMSVDSRFRGKSIAKALGRRVLEFAMLHNYSAVVLGTTAVKVAAHKLYESLGFRHMGASDHYVLPGMTLSLAERLFFQVRYHRYRLQLREE

SEQ ID NO: 7 - protein, 299 aa, Rattus sp.
MHCGPPDMVCETKIVATEDHEALPGAKKDALLAAAGAMWPPLPAAPGPAAAPPPAAGPQPHGGTGGAGPPEGRGVCIREFRAAEQEAARRIFYDGILERIPNTAFRGLRQHPRTQLLYALLAALCFAVTRSLLLTCLVPAGLLALRYYYSRKVILAYLECALHTDMADIEQYYMKPPGSCFWVAVLDGNVVGIVAARAHEEDNTVELLRMSVDSRFRGKGIAKALGRRVLEFAMLHNYSAVVLGTTAVKVAAHKLYESLGFRHMGASDHYVLPGMTLSLAERLFFQVRYHRYRLQLREE

SEQ ID NO: 8 - protein, 271 aa, Xenopus tropicalis
MTYRGTRKSPCCSPPPRCGPPLPSGPAGSALGPPSSGAEEEMTKEQVYLREFQPADQEFARRIFYEGIKERILSSAFRGLKYQPLLQSVYAVIIIMCFVVTKSLLVTCCMPLFLLGMRYYYSRKIILNHLECALRTDMSDIEQYYMKQPGSCFWVAVLEGKVVGIVAARGNEEDNVVELRRMSVDSNYRGKGIAKALGRKVLEFAMLNHYSSIVLGTTAVKIAAHKLYESLGFKHVGVVEHHIVPGMTHSLLERLFFQLRYHRYCLQLREE

SEQ ID NO: 9 - protein, 282 aa, Danio rerio
MHCSSPKMVCETKIVADEHEAIAGTKKDSIIVSSSQMWTSSSASPSALESKIEKRNQVFIREFERSDHEEVRRIFNEGIMERIPNSAFRGLKQQTTTQFMYAFLTVMCYVMTKSFTLTFCAPFILMGARYYYSRKVILSYLDCALHTDMADIEAYYMKPTGSCFWVAVLQGQVVGIVAAQSREDDNTVELRRMSVDSHFRGKGIAKALGRRVIEFAMLNNYSAVVLGTTAVKMAAHKLYESLGFRRVGETEDYTLPGMTRSPLERLFFQIRYSHYRLQLHEE

SEQ ID NO: 10 - protein, 172 aa, Bacillus subtilis
MSVKMKKCSREDLQTLQQLSIETFNDTFKEQNSPENMKAYLESAFNTEQLEKELSNMSSQFFFIYFDHEIAGYVKVNIDDAQSEEMGAESLEIERIYIKNSFQKHGLGKHLLNKAIEIALERNKKNIWLGVWEKNENAIAFYKKMGFVQTGAHSFYMGDEEQTDLIMAKTLI

SEQ ID NO: 11 - protein, 16 aa, Homo sapiens
KIVAAEDHEALPGAKK

SEQ ID NO: 12 - protein, 12 aa, Homo sapiens
RRIFYDGIMERI

SEQ ID NO: 13 - protein, 11 aa, Homo sapiens
RIFYDGIMERI

SEQ ID NO: 14 - protein, 9 aa, Homo sapiens
RIPNTAFRG

SEQ ID NO: 15 - protein, 14 aa, Homo sapiens
RAHEEDNTVELLRM

SEQ ID NO: 16 - protein, 10 aa, Homo sapiens
KLYESLGFRH

SEQ ID NO: 17 - protein, 8 aa, Homo sapiens
RLFFQVRY

(19) United States
(12) Patent Application Publication
(10) Pub. No.: US 2018/0340156 A1
(43) Pub. Date: Nov. 29, 2018

(54) PURIFICATION OF A SOLUBLE AND ACTIVE FORM OF ASPARTATE N-ACETYLTRANSFERASE

(71) Applicant: The University of Toledo, Toledo, OH (US)

(72) Inventors: Ronald E. Viola, Toledo, OH (US); Qinzhe Wang, Toledo, OH (US); Mojun Zhao, Toledo, OH (US); Gwenn Parungao, Toledo, OH (US)

(73) Assignee: The University of Toledo, Toledo, OH (US)

(21) Appl. No.: 15/757,837

(22) PCT Filed: Sep. 8, 2016
(86) PCT No.: PCT/US2016/050737

§ 371 Date: Mar. 6, 2018

Related U.S. Application Data
(60) Provisional application No. 62/216,700, filed on Sep. 10, 2015.

(51) Int. Cl. C12N 9/10; C12N 9/02; C07K 14/245; C12Q 1/48

(52) U.S. Cl. CPC C12N 9/1029; C12N 9/0051; C12Q 1/48; C12N 9/1088; C07K 14/245; C12Y 108/01009

(57) ABSTRACT

Fusion proteins that include N-acetylaspartate synthetase (ANAT) and at least one solubilizing partner, such as glutathione 5-transferase (GST), thioredoxin (TRX), or maltose binding protein (MBP), are described. Also described are methods of making the fusion proteins, methods of solubilizing the fusion proteins, methods of purifying the fusion proteins, and methods of using the fusion proteins.

### RELATED APPLICATIONS

This application claims priority to U.S. Provisional Application Ser. No. 62/216,700 filed under 35 U.S.C. § 111(b) on Sep. 10, 2015, the disclosure of which is incorporated herein by reference in its entirety.

### STATEMENT REGARDING FEDERALLY SPONSORED RESEARCH

This invention was made with no government support. The government has no rights in this invention.

### SEQUENCE LISTING

The instant application contains a Sequence Listing which has been submitted electronically in ASCII format and is hereby incorporated by reference in its entirety. Said ASCII copy, created on Sep. 8, 2016, is named 53-57357-D2016-26_SL.txt and is 16,329 bytes in size.

### BACKGROUND OF THE INVENTION

Canavan disease (CD) is a fatal, neurological disease that is caused by an interruption in the metabolism of a critical amino acid, N-acetyl-L-aspartic acid (NAA). Aspartoacylase (ASPA), the responsible enzyme, specifically catalyzes the hydrolysis of NAA, releasing L-aspartate and acetate. (FIG. 1.) While the enzymatic activity, protein structure, and clinical mutants of aspartoacylase have been characterized, little is known about the NAA synthetic enzyme.

Defects at multiple locations in the aspA gene that codes for ASPA lead to mutant forms of this enzyme that are either not expressed or rapidly degraded, or have significantly impaired catalytic activity. As a consequence, the substrate NAA accumulates to unusually elevated levels and the products of this reaction, aspartic acid and acetate, are not made in oligodendrocytes. Various hypotheses of the molecular basis for CD have been proposed and tested, including toxic accumulation of NAA, increased NAAG neurotransmitter production, osmotic effects of NAA accumulation, and defects in fatty acid biosynthesis as a consequence of acetate deficiency.

Animal models in which the aspA gene had been knocked out were found to reproduce many of the disease symptoms. Unexpectedly, when a second gene knock-out was introduced, in the Nat8l gene which codes for the enzyme that synthesizes NAA, these effects were reversed, leading to normal myelination and a decrease in CD symptoms. These results indicate that Canavan symptoms can be reduced by lowering NAA levels, and place an increased importance on the need to characterize aspartate N-acetyltransferase (ANAT), the brain enzyme that catalyzes the synthesis of NAA, for the development of potent ANAT inhibitors. Unfortunately, prior attempts to express and purify this enzyme have resulted in only limited progress. This enzyme has been shown to be membrane-associated, with a structural model indicating the presence of a membrane anchor region. Attempts to extract a functional enzyme from membrane preparations have not succeeded, thwarted by at least the apparent sensitivity of this enzyme to the presence of detergents. Thus, there is a need in the art for methods of extracting and/or purifying a functional aspartate N-acetyltransferase enzyme that will allow for inhibitors to be developed.

### SUMMARY OF THE INVENTION

Provided herein is a fusion protein comprising an amino acid construct between an N-terminus and a C-terminus, the amino acid construct containing N-acetylaspartate synthetase (ANAT) and at least one solubilizing partner. The amino acid construct can optionally include one or more polyhistidine (his) tags. In certain embodiments, the solubilizing partner is selected from the group consisting of glutathione S-transferase (GST), thioredoxin (TRX), and maltose binding protein (MBP). In certain embodiments, the fusion protein includes a polyhistidine tag at the C-terminus. In certain embodiments, the fusion protein includes a polyhistidine tage at the N-terminus. In certain embodiments, the fusion protein includes a polyhistidine tage at both the C-terminus and the N-terminus. In certain embodiments, the amino acid construct consists essentially of ANAT and one or more solubilizing partners.

In certain embodiments, the fusion protein comprises a MBP-ANAT sequence. In certain embodiments, the amino acid construct consists essentially of MBP-ANAT.

In certain embodiments, the fusion protein comprises a TRX-his-ANAT-his sequence. In certain embodiments, the amino acid construct consists essentially of TRX-his-ANAT-his.

In certain embodiments, the fusion protein comprises a GST-his-ANAT-his sequence. In certain embodiments, the amino acid construct consists essentially of GST-his-ANAT-his.

In certain embodiments, the fusion protein comprises a his-MBP-ANAT-his sequence. In certain embodiments, the amino acid construct consists essentially of his-MBP-ANAT-his.

In certain embodiments, the fusion protein retains enzymatic activity of human ANAT.

In certain embodiments, the fusion protein is solubilized in a detergent. In particular embodiments, the fusion protein is soluble at concentrations up to 6 mg/ml. In particular embodiments, the detergent is selected from the group consisting of Triton X-100, SDS, C₁₂E₈, Tween 20, DDM, and Cymal 5. In particular embodiments, the detergent is present at or above the critical micelle concentration of the detergent. In particular embodiments, the fusion protein retains at least 50% enzymatic activity of human ANAT.

In certain embodiments, the fusion protein is stable at temperatures ranging from −80° C. to room temperature. In certain embodiments, the fusion protein binds to L-aspartate or acetyl-CoA.

Further provided is a variant fusion protein comprising an amino acid sequence having at least about 75% sequence identity to a fusion protein described herein. Further provided is a variant fusion protein comprising an amino acid sequence having at least about 85% sequence identity to a fusion protein described herein. Further provided is a variant fusion protein comprising an amino acid sequence having at least about 90% sequence identity to a fusion protein described herein. Further provided is a variant fusion protein comprising an amino acid sequence having at least about 95% sequence identity to a fusion protein described herein.

Further provided is a method of constructing a fusion protein, the method comprising constructing a polyhistidine-tagged fusion protein having an amino acid construct containing ANAT and a solubilizing partner, and exposing the polyhistidine-tagged fusion protein to a mutagen to produce a fusion protein. In certain embodiments, the solubilizing partner is selected from the group consisting of GST, TRX, and MBP. In certain embodiments, the method further comprises purifying the fusion protein by metal-affinity chromatography. Also provided is the product of the method.

Further provided is a method of purifying N-acetylaspartate synthetase (ANAT), the method comprising constructing a fusion protein by fusing ANAT with at least one solubilizing partner, and subjecting the fusion protein to Ni-immobilized metal affinity chromatography to purify the fusion protein. In certain embodiments, the at least one solubilizing partner is selected from the group consisting of glutathione S-transferase (GST), thioredoxin (TRX), and maltose binding protein (MBP). In certain embodiments, the Ni-immobilized metal affinity chromatography comprises a tandem affinity process involving a Ni-NTA column and a second affinity chromatography step that takes advantage of the binding properties of any fusion protein partner, such as an amylose column when MBP is present in the amino acid construct. Also provided is the product of the method.

Further provided is a method of solubilizing N-acetylaspartate synthetase (ANAT), the method comprising constructing a fusion protein by fusing ANAT with at least one solubilizing partner, and incubating the fusion protein in a detergent at or above the critical micelle concentration to solubilize the fusion protein. In certain embodiment, the at least one solubilizing partner is selected from the group consisting of glutathione S-transferase (GST), thioredoxin (TRX), and maltose binding protein (MBP). In certain embodiments, the detergent is selected from the group consisting of Triton X-100, SDS, C₁₂E₈, Tween 20, DDM, and Cymal 5. Also provided is the product of the method.

Further provided is a method of developing a treatment for Canavan disease, the method comprising fusing ANAT with at least one solubilizing partner, to create a soluble ANAT fusion protein, and testing inhibitor candidates against the soluble ANAT fusion protein to develop a treatment for Canavan disease. In certain embodiments, the solubilizing partner is selected from the group consisting of glutathione S-transferase (GST), thioredoxin (TRX), and maltose binding protein (MBP).

Further provided is a method for making a fusion protein, the method comprising the steps of cloning a human nat8l gene into a plasmid, wherein the plasmid contains a gene for at least one solubilizing partner; transforming E. coli cells with the plasmid; growing the E. coli cells for a period of time; and inducing protein expression in the E. coli cells to produce a fusion protein. In certain embodiments, the at least one solubilizing partner is selected from the group consisting of thioredoxin (TRX), glutathione S-transferase (GST), or maltose binding protein (MBP).

Further provided is a kit for creating a fusion protein, the kit comprising a first container housing a plasmid containing the human nat8l gene and a gene for at least one of glutathione S-transferase (GST), thioredoxin (TRX), or maltose binding protein (MBP), and a second container housing E. coli cells.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: Diagram showing NAA metabolism.

FIG. 2: Different fusion constructs of human aspartate N-acetyltransferase (ANAT). The DNA lengths are drawn roughly to scale, with the nat8l gene shown in green, and the 6×his tag (SEQ ID NO: 2) in yellow. The thioredoxin (TRX) fusion partner is in red, the glutathione S-transferase (GST) is in orange, and the maltose binding protein (MBP) is in blue.

FIGS. 3A-3C: Purification of ANAT via a tandem affinity approach: Ni-NTA (FIG. 3A) followed by amylose column (FIG. 3B). FIG. 3C shows SDS-PAGE (left) and Western blot (right) of the purification of the MBP-ANAT-his enzyme construct. Lane 1: Ni-IMAC purification; Lane 2: Amylose affinity purification.

FIGS. 4A-B: Illustration of an enzymatic assay used to test for ANAT activity (FIG. 4A) and graph of enzyme activity as a function of substrate concentration (FIG. 4B). The assay comprises 8 mM potassium phosphate, 4 mM Tris-HCl, 120 mM NaCl, 4% glycerol, 40 μM DTNB, 40 μM acetyl-CoA, and 2 mM L-aspartate at pH 7.4 in a total volume of 200 μl.

FIG. 5: Sequence alignment of human aspartate N-acetyltransferase (ANAT) (SEQ ID NO: 1) with closely related ANATs from mouse (SEQ ID NO: 6), rat (SEQ ID NO: 7), western clawed frog (SEQ ID NO: 8), and zebra fish (SEQ ID NO: 9), and a related bacterial spermidine acetyltransferases from Bacillus subtilis (SEQ ID NO: 10). The membrane anchor region in the mammalian enzymes is highlighted in yellow, and the turn region in the soluble bacterial enzyme is shown in red. Without wishing to be bound by theory, it is believed ANAT contains a long hydrophobic region.

FIGS. 6A-6B: Effect of potential inactivators on enzyme activity. FIG. 6A shows an examination of different detergents tested at their CMC concentrations. FIG. 6B shows the effect of increasing DMSO concentration on catalytic activity. Error bars are shown for values determined in duplicate or triplicate.

FIG. 7: Vₘₐₓ/Kₘ pH profile of the MBP-ANAT fusion enzyme. The loss of activity with decreasing pH was fitted to a model (dashed line) that reflects the protonation of a functional group with a pK of 6.8.

FIGS. 8A-8C: Screen shots showing the confirmation of ANAT. FIG. 8A shows the identification of ANAT protein by MALDI-MS. FIG. 8B shows six peptides were mapped to ANAT.

FIG. 8B discloses SEQ ID NOS. 1 and 11-17, respectively, in order of appearance. FIG. 8C shows peptide sequencing results. FIG. 8C discloses SEQ ID NO: 4.

FIG. 9: Table 1, showing detergent extraction of native Aspartate N-acetyltransferase.

FIG. 10: Table 2, showing substrate kinetic parameters for Aspartate N-acetyltransferase.

### DETAILED DESCRIPTION OF THE INVENTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Canavan disease is caused by an interruption in the metabolism of N-acetylaspartate (NAA). Numerous mutations have been found in the enzyme that hydrolyzes NAA, and the catalytic activity of aspartoacylase is significantly impaired in CD patients. It is believed that the enzyme that catalyzes the synthesis of NAA in the brain plays an important role in CD. However, previous attempts to study this enzyme have not succeeded in obtaining a soluble, stable, and active form of this membrane-associated protein. In accordance with the present disclosure, fusion constructs with solubilizing protein partners have been created to obtain an active and soluble form of aspartate N-acetyltransferase (ANAT). A wide range of detergents, properties, and conditions were surveyed to accomplish extraction of the ANAT enzyme from membranes. Coupling this approach with the production of a fusion construct with a solubilizing protein partner has led to the successful extraction and purification of active ANAT. The catalytic properties of this purified enzyme have been examined, along with its substrate specificity and optimized stabilization conditions. Selective inhibitors that can lower the elevated levels of NAA that are observed in CD patients can now be developed using this active, soluble form of the enzyme, opening doors to new treatment therapies.

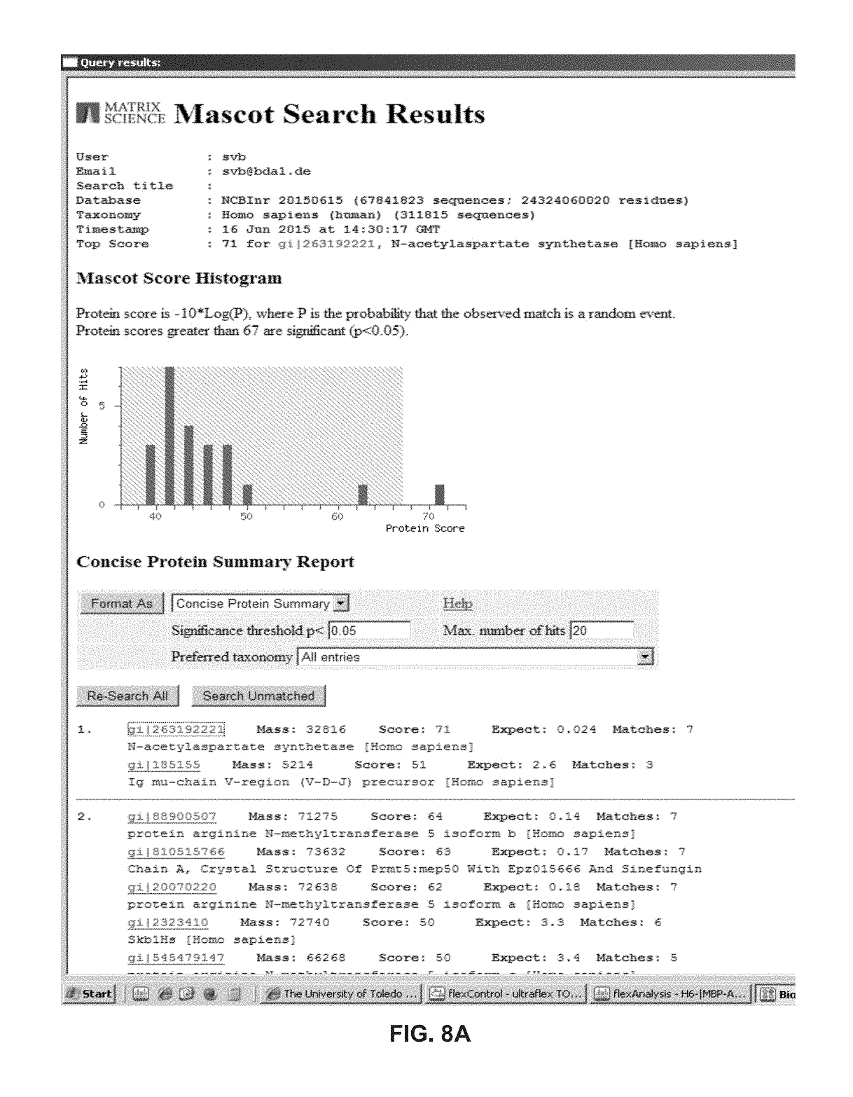

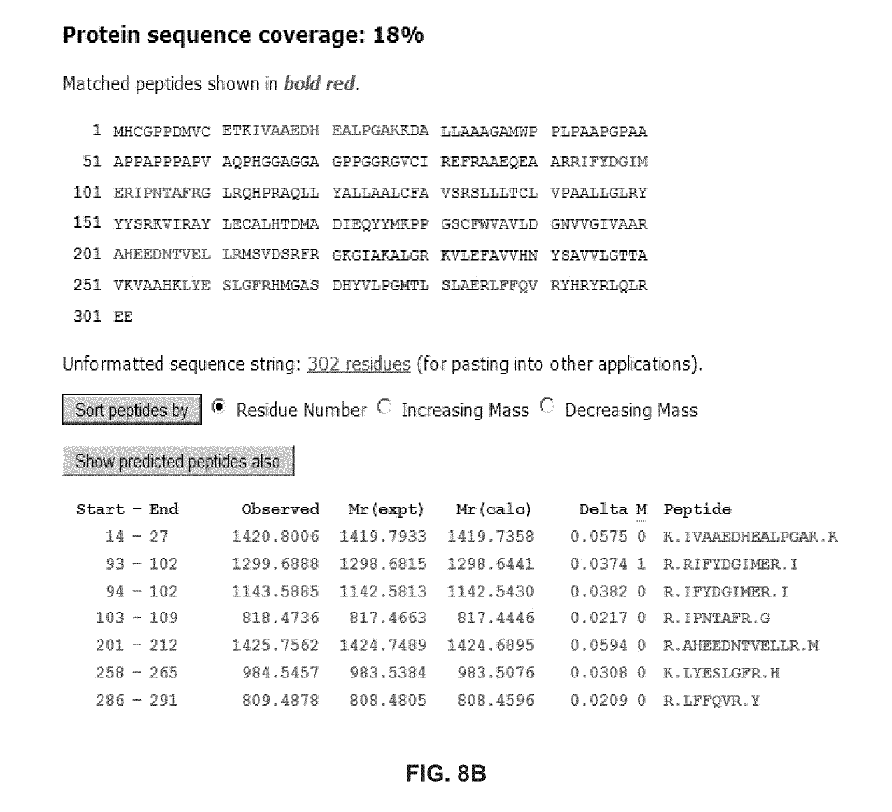

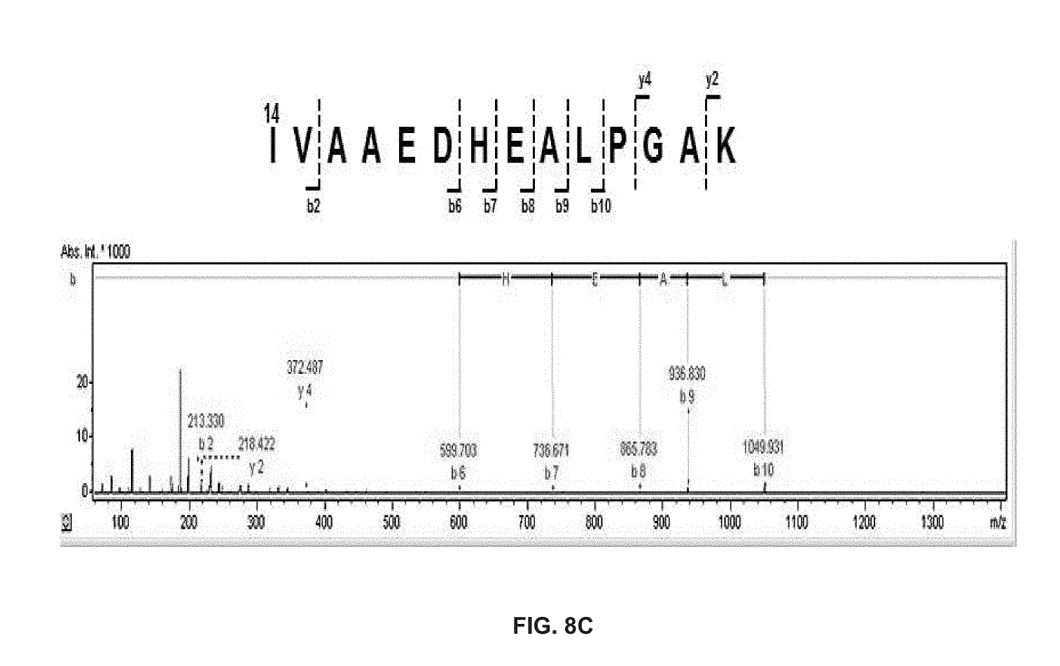

Provided herein are fusion proteins having amino acid sequences which contain N-acetylaspartate synthetase (ANAT). ANAT has the amino acid sequence of

[SEQ ID NO: 1]
MHCGPPDMVCETKIVAAEDHEALPGAKKDALLAAAGAMWPPLPAAPGPAA
APPAPPPAPVAQPHGGAGGAGPPGGRGVCIREFRAAEQEAARRIFYDGIM
ERIPNTAFRGLRQHPRAQLLYALLAALCFAVSRSLLLTCLVPAALLGLRY
YYSRKVIRAYLECALHTDMADIEQYYMKPPGSCFWVAVLDGNVVGIVAAR
AHEEDNTVELLRMSVDSRFRGKGIAKALGRKVLEFAVVHNYSAVVLGTTA
VKVAAHKLYESLGFRHMGASDHYVLPGMTLSLAERLFFQVRYHRYRLQLR
EE
(FIG. 8B).

The term “fusion protein” refers to a protein having two or more portions covalently linked together, where each of the portions is derived from different proteins or sequences. In the present disclosure, the fusion proteins are constructs of ANAT with one or more solubilizing partners, and optionally one or more polyhistidine tags. In certain embodiments, the fusion proteins are soluble, active forms of human ANAT. A wide range of solubilizing partners can be used. In certain non-limiting examples, the solubilizing partners are selected from glutathione S-transferase (GST), thioredoxin (TRX), or maltose binding protein (MBP). More than one solubilizing partner, including multiple instances of the same solubilizing partner, can be present. The creation of protein constructs with solubilizing partners has led to higher solubility and increased stability of the ANAT enzyme.

Amino acid sequence variants of the fusion proteins are encompassed within the present disclosure. Modifications to the fusion proteins can be introduced by peptide synthesis. Such modifications include, for example, deletions from, insertions into, and/or substitutions within the amino acid sequence of ANAT and/or the solubilizing partner(s) present. Any combination of deletion, insertion, and substitution can be made to arrive at the final amino acid construct of the fusion protein, provided that the final construct possesses the desired solubility and biological activity, such as the enzymatic activity of human ANAT. Accordingly, provided herein are variants of the fusion proteins described. In some embodiments, the fusion protein includes an ANAT variant having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of human ANAT. In some embodiments, the fusion protein includes a variant of the solubilizing partner, such as a variant of glutathione S-transferase (GST), thioredoxin (TRX), or maltose binding protein (MBP), having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the solubilizing partner. In some embodiments, the fusion protein is a variant fusion protein having an amino acid sequence having at least about 75%, or at least about 85%, or at least about 90%, or at least about 95% sequence identity to the amino acid construct of ANAT fused with a solubilizing partner, such as GST, TRX, or MBP. Reference to a “% sequence identity” with respect to a reference polypeptide is defined as the percentage of amino acid residues in a candidate sequence that are identical with the amino acid residues in the reference polypeptide, after aligning the sequences and introducing gaps, if necessary, to achieve the maximum percent sequence identity, and not considering any conservative substitutions as part of the sequence identity.

The fusion proteins described herein are stable and soluble in various detergents, such as non-ionic, ionic, or zwitterionic detergents. Suitable detergents for solubilizing the fusion proteins include, but are not limited to, Triton X-100, SDS, C₁₂E₈, Tween 20, DDM, and Cymal 5. In certain embodiments, the fusion proteins can be solubilized in a detergent at concentrations up to 6 mg/ml without significant precipitation or loss of enzymatic activity.

Methods of screening for ANAT inhibitors, and thereby developing treatments for Canavan disease, are encompassed within the present disclosure. The active, soluble form of ANAT provided by the fusion proteins containing ANAT and a solubilizing partner allow for the evaluation of possible inhibitors such as, but not limited to, small molecules, peptides, and nucleic acids.

It is also envisioned that the fusion proteins and methods described herein can be embodied as parts of a kit or kits. A non-limiting example of such a kit is a kit for making a fusion protein as described herein, which includes a plasmid containing the human nat8l gene and a gene for at least one of glutathione S-transferase (GST), thioredoxin (TRX), or maltose binding protein (MBP), and E. coli cells in separate containers, where the containers may or may not be present in a combined configuration. Many other kits are possible. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive, CD-ROM, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

Materials

5,5′-Dithiobis(2-nitrobenzoic acid) (DTNB) was obtained from Sigma. Detergents were obtained from Anatrace, and the protease inhibitor cocktail (P8340) was obtained from Sigma-Aldrich. NiCo21 (DE3) competent E. coli cells were from New England Biolabs. A modified pET28a plasmid with the incorporation of a human rhinovirus 3C protease cleaving site was a gift from Dr. Don Ronning (University of Toledo).

Expression and Solubilization of Native ANAT

The human nat8l gene was cloned into the pETDEST42 plasmid using the Gateway cloning technology (Life Technologies). This plasmid was used to transform E. coli BL21(DE3) cells for protein production. The cells were grown at 37° C. to an A₆₀₀ of 0.6-0.8 in Luria-Bertani (LB) medium containing 100 μg/mL ampicillin, and gene expression was then induced with 1 mM isopropyl α-D-thiogalactopyranoside (IPTG) at 28° C. for 5 hours. The cell pellet was resuspended in buffer A [50 mM HEPES, 500 mM NaCl and 10% glycerol (pH 7.4)] and lysed by sonication on ice for 8 min with 30 s pulse on and 2 min pulse off cycle. The cell lysate was centrifuged at 11,000 rpm for 30 minutes to pellet cell debris and unlysed cells. The membrane fraction was collected by high-speed centrifugation of the supernatant at 40,000 rpm for 1 h. Solubilization trials of ANAT from the membrane fraction were conducted with a variety of different detergents that include ionic, non-ionic, and zwitterionic detergents. Twenty microliter aliquots of the resuspended membrane fraction were mixed with different amounts of stock detergent solutions each prepared at 10 times their critical micelle concentration (CMC), leading to a final concentration of 1.5×CMC and 8.7×CMC. These solutions were incubated at room temperature for 1 h or at 4° C. overnight in a rocking platform. The supernatant was separated from the pellet by centrifugation and the proteins that were extracted into the supernatant were analyzed by SDS-PAGE and Western blotting.

Expression and Purification of ANAT Fusion Enzymes

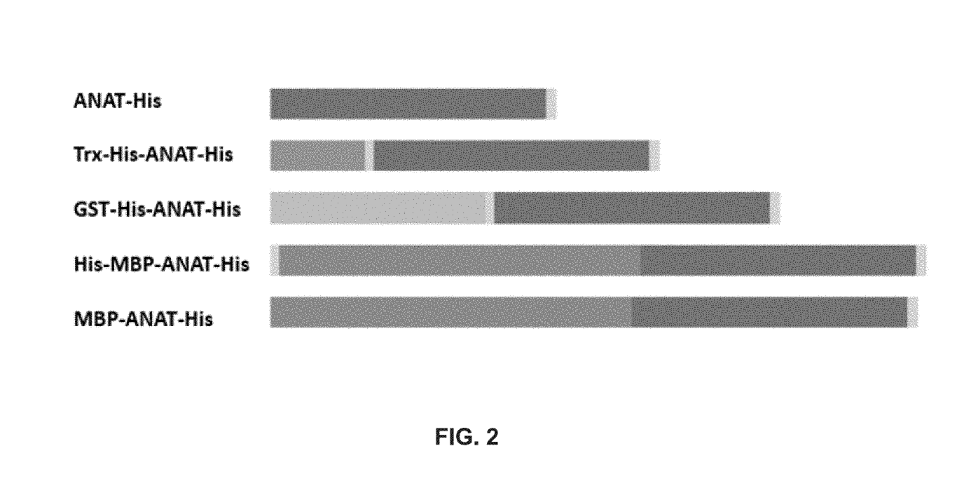

The E. coli codon optimized gene was inserted into the modified pET28 plasmid using the EcoRI and XhoI restriction sites. Different versions of this plasmid have been used to incorporate the genes for either thioredoxin (TRX), glutathione S-transferase (GST), or maltose binding protein (MBP), followed by a 21 to 68-amino acid cleavage site sequence and linker to the N-terminal of the nat8l gene (FIG. 2). These fusion constructs also contain polyhistidine tags located at both the N- and C-termini of each fusion protein. ANAT was expressed in E. coli BL21(DE3) cells grown in LB media. The culture was grown at 37° C. until it reached an A₆₀₀ of between 0.6 and 0.8, followed by induction with IPTG at 30° C. for 5 h. The recombinant fusion enzymes were purified by using Ni-immobilized metal affinity chromatography (IMAC).

Expression and Dual Affinity Purification of MBP-ANAT Fusion Enzyme

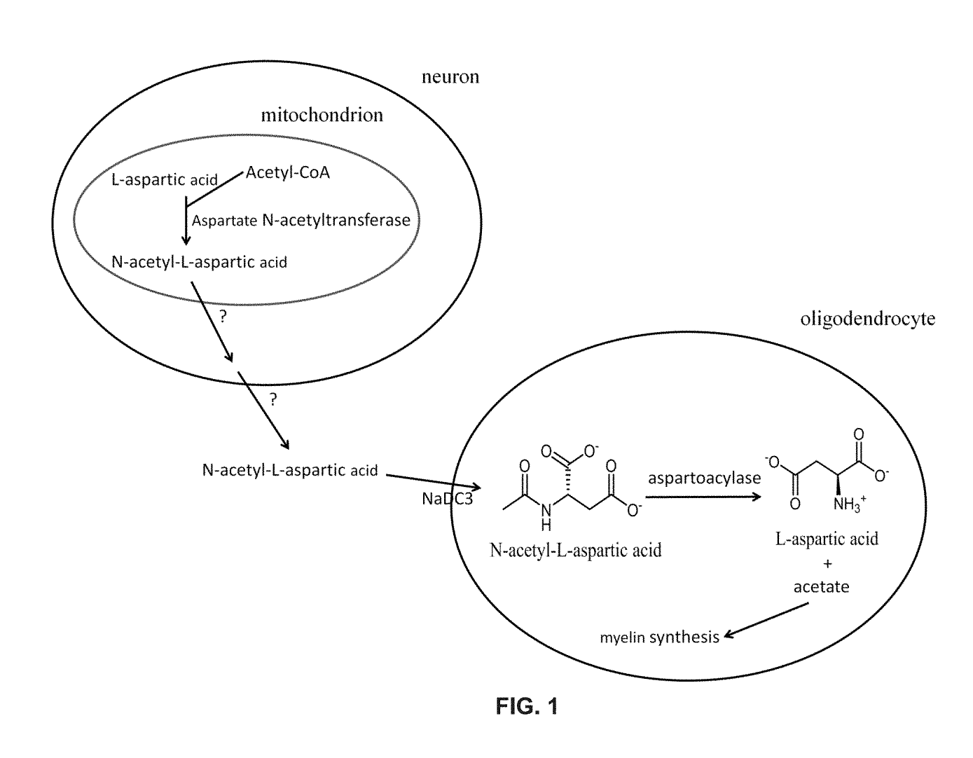

The MBP-ANAT-his construct (FIG. 1) was produced from the his-MBP-ANAT-his construct by mutagenesis. NiCo21(DE3) competent E. coli cells containing the MBP-ANAT-his construct were selected on LB plates with 30 μg/ml kanamycin at 37° C. for 16 hours. Colonies from these plates were used to inoculate starter cultures containing 10 ml of LB media. After 16 hours growth at 37° C., each starter culture was diluted 100-fold into 1 L of LB media, and cell growth was continued for about 2 hours until A₆₀₀ reached 0.6. IPTG was then added to a final concentration of 0.5 mM and protein expression was induced at 16° C. for 20 hours. The dual affinity tagged human ANAT was initially purified by metal-affinity chromatography (IMAC). The column was washed with Buffer A, containing 20 mM potassium phosphate, pH 7.4, 300 mM sodium chloride, 10% glycerol, and 20 mM imidazole, and then eluted with a linear gradient of Buffer B (buffer A containing 400 mM imidazole). The active fractions were pooled and loaded onto an amylose column and highly purified ANAT fusion was then obtained by elution with a 0 to 10 mM linear maltose gradient (FIG. 2). Protein concentration was determined by NanoDrop 2000 UV-Vis Spectrophotometer (Thermo Scientific), and enzyme activity was measured as described below.

Enzyme Activity Assay

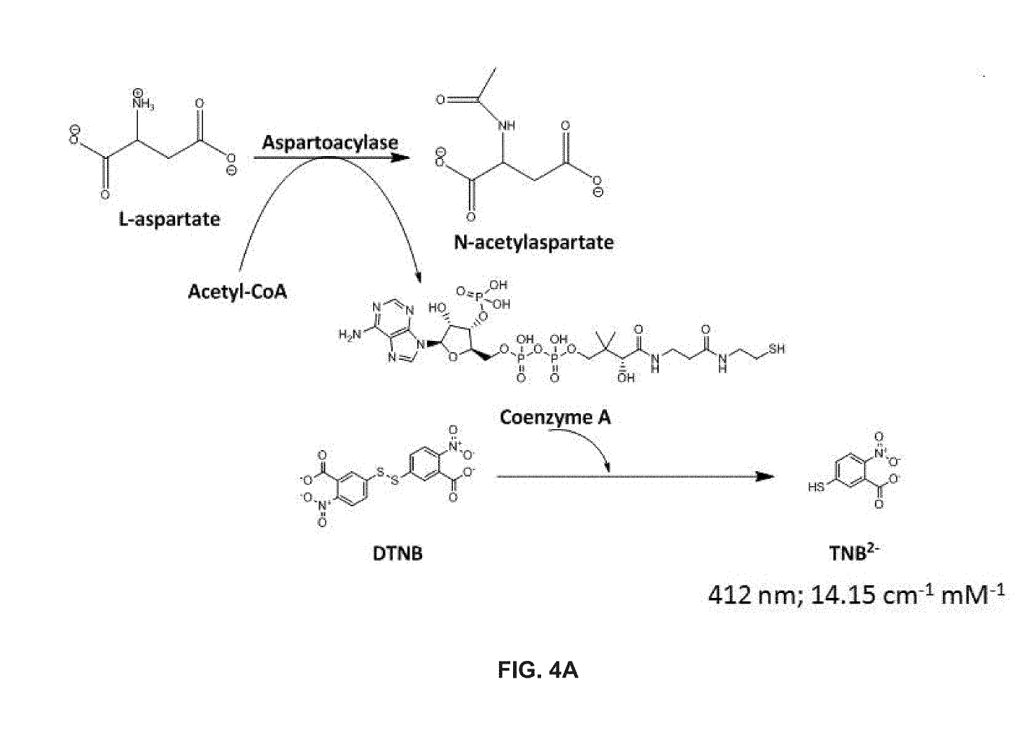

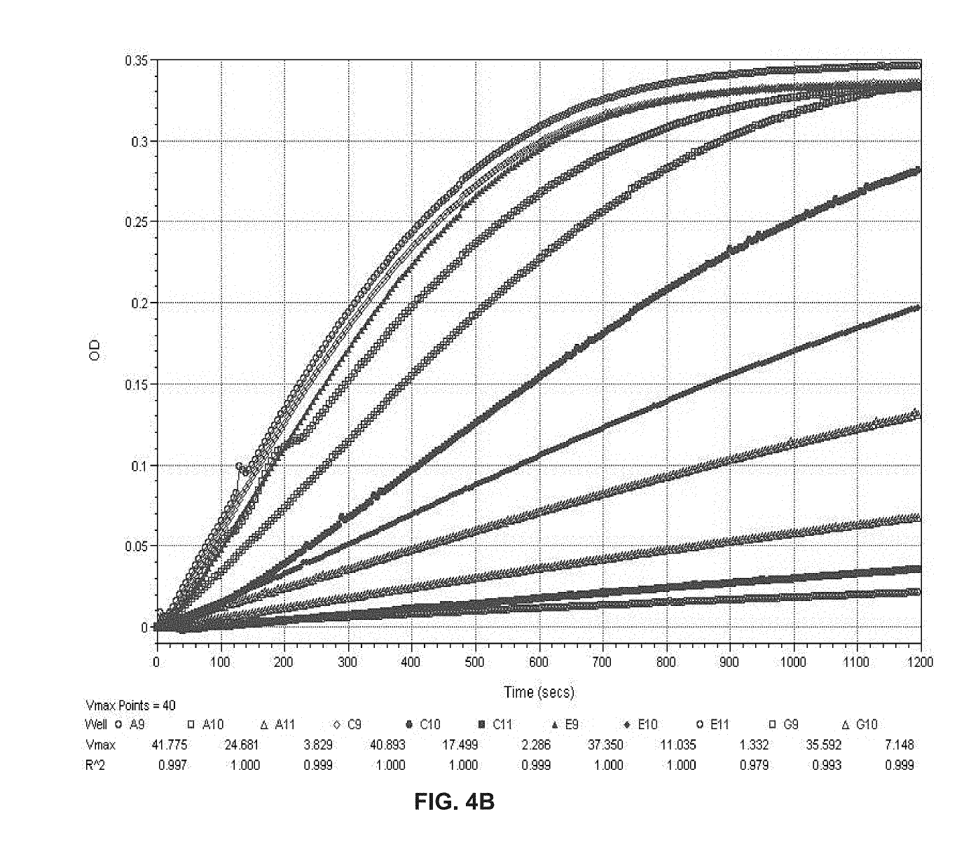

The ANAT activity was measured by an established DTNB-based assay in which the coenzyme A product participates in a thiol exchange reaction with DTNB. This assay is illustrated in FIGS. 4A-4B. The resulting TNB²⁻ ion was monitored at 412 nm (c=14.15 mM⁻¹cm⁻¹) using a SpectraMax 190 spectrophotometer plate reader (Molecular Devices, CA). A typical assay contains 8 mM potassium phosphate, 4 mM Tris-HCl, 120 mM NaCl, 4% Glycerol, 40 μM DTNB, 40 μM acetyl-CoA, 2 mM L-aspartate at pH 7.4 in a total volume of 200 μl. The results of the DTNB-based assay were validated by an HPLC end point assay that directly measures the coenzyme A product at 260 nm.

Enzyme Kinetic and Stability Studies

The DTNB-based assay was used to measure the kinetic parameters for the physiological substrates and some alternative ANAT substrates. The reaction rates were measured with varying substrate concentrations of L-aspartate (0.01 to 2 mM) and acetyl-CoA (2.5 to 50 μM), and the data were fitted to the equation for a sequential enzyme mechanism (eq. 1) to determine the kinetic parameters:

$$velocity = \frac{{V}_{max} [ A ] [ B ]}{{K}_{a} [ B ] + {K}_{b} [ A ] + [ A ] [ B ] + {K}_{ia} {K}_{b}} \qquad ( eq . 1 )$$

where Kₐ and K_{b} are the Michaelis constants, and Kᵢₐ is the binding constant for substrate A.

To examine the effects of pH on enzyme activity, rates were measured at pH values from 6.0 to 9.5 by varying the L-aspartate concentration at fixed, saturating levels of acetyl CoA. The Vₘₐₓ and Vₘₐₓ/Kₘ values determined at each pH were fitted to an equation for the protonation of a group on the enzyme that causes the loss of catalytic activity (eq. 2):

log Vₘₐₓ(or Vₘₐₓ/Kₘ)=log [C/(1+[H]/Kₐ)] (eq. 2)

where C is the pH independent rate and Kₐ is the equilibrium constant for protonation of a group.

The effect of detergent properties on ANAT activity was tested using a variety of different detergents, each at their critical micelle concentration (CMC) levels. All measurements were conducted at least in duplicate by using the DTNB-based assay at saturating substrates levels. The effect of an organic solvent on ANAT activity was also evaluated by varying the DMSO concentration in the assay from 0 to 50%. To test the stability of the purified MBP-ANAT-his construct, the enzyme was stored at 4° C. for several days and activity was monitored daily. To test for the optimal long term storage conditions, the enzyme activity was followed through multiple freeze-thaw cycles between −80° C. and room temperature.

Results

Enzyme Production

Human L-aspartate N-acetyltransferase (ANAT) was previously identified as a membrane-associated protein. Detergents such as Triton X-100 and CHAPS had been used in an attempt to extract ANAT from tissue. However, inefficient solubilization and detergent sensitivity limited the effectiveness of this approach. Now, three different approaches have been examined to overcome the previous barriers to the production of stable and active human ANAT: (1) partial removal of the putative membrane anchor region; (2) extensive screening of detergent solubilization conditions for the native enzyme; and (3) formation of fusion constructs with solubilizing protein partners.

Membrane Anchor Removal

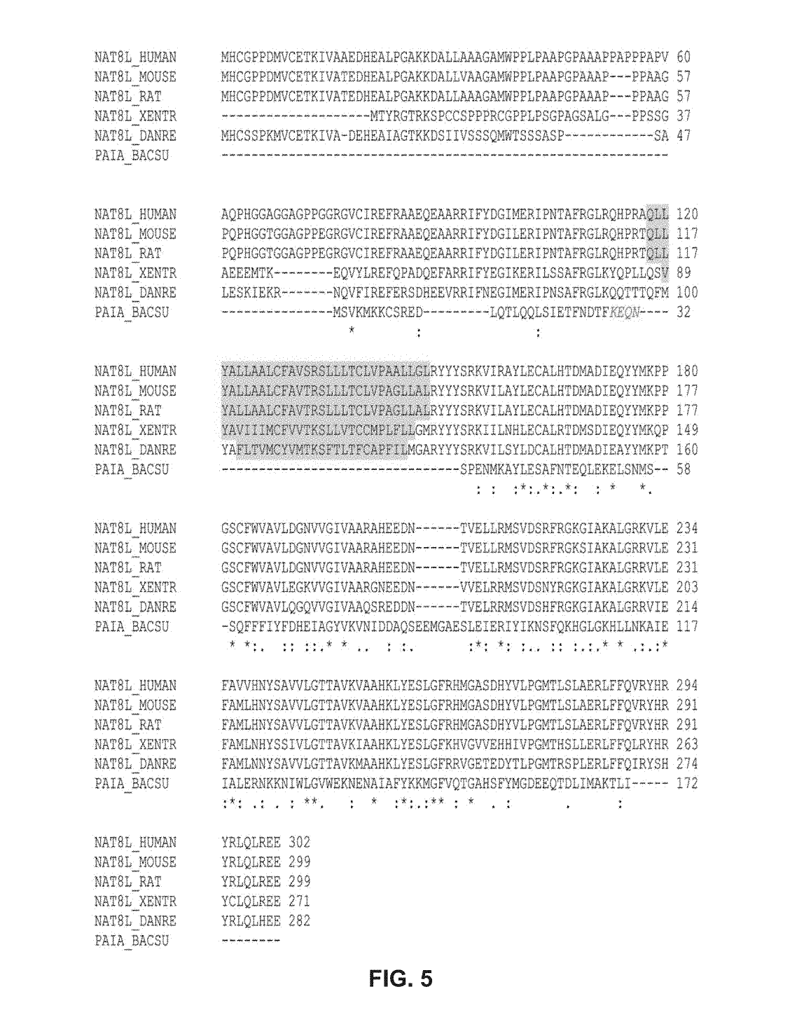

ANAT is believed to have a 30-amino acid hydrophobic helix-turn-helix region, based on a domain model produced from a sequence alignment between human ANAT and a homologous enzyme, polyamine N-acetyltransferase (PaiA) from B. subtilis (FIG. 5). This model of ANAT was guided by the published structure of PaiA, and the effects of point mutations in different domains has provided some experimental support for this structural model. The predicted hydrophobic helixes in the membrane anchor region of the human enzyme are replaced by a short loop in the soluble bacteria N-acetyltransferase. This comparison indicates that a soluble and active form of human ANAT could be produced through protein engineering of this region. To test this approach, the hydrophobic helical region in human ANAT was replaced by a short peptide, KEQN (SEQ ID NO: 3), which corresponds to the amino acid sequence in the turn region that is present in the soluble PaiA structure (FIG. 5). Unfortunately, this engineered protein is still found to be present predominately in the membrane fraction, based on Western blot analysis, and fractions from a preliminary Ni-IMAC purification of a detergent-solubilized protein sample did not yield any detectable ANAT activity.

Detergent Solubilization

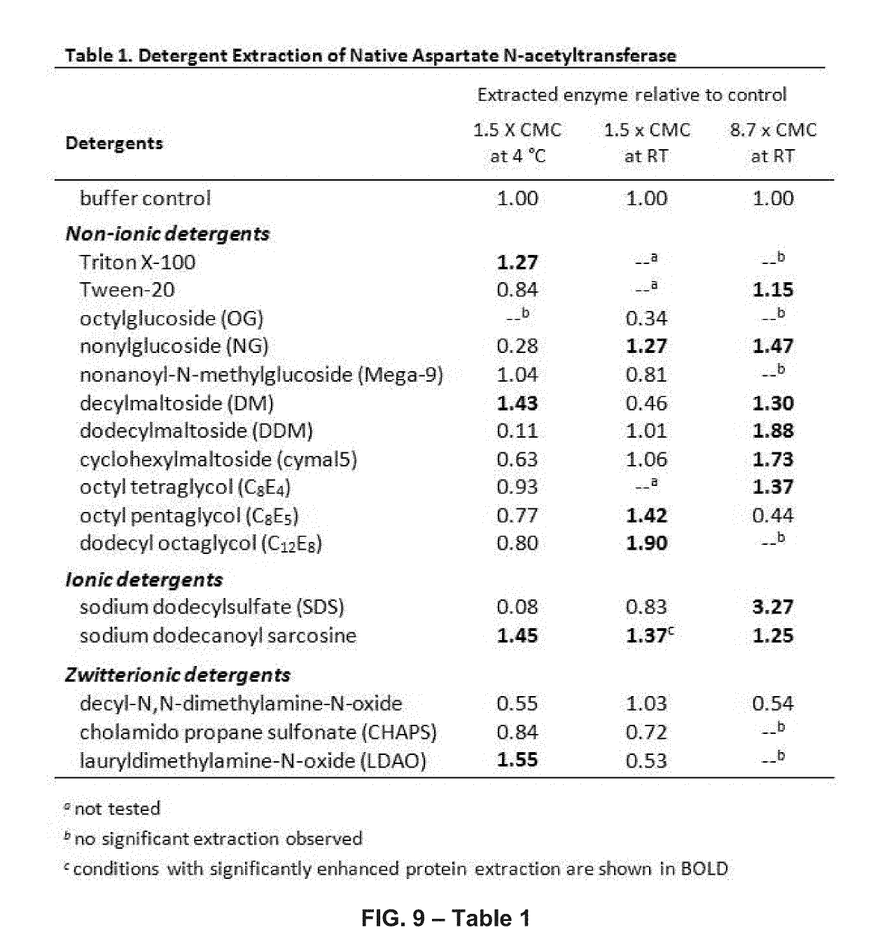

To find suitable conditions for the efficient extraction of native ANAT, membrane solubilization tests were performed using detergents with a range of structural properties. As seen in Table 1 (FIG. 9), the ionic detergent SDS is the most efficient detergent in membrane solubilization and extraction of the membrane-bound ANAT. Among the non-ionic detergents, C₁₂E₈ showed the highest extraction efficiency at lower detergent concentrations (1.5 CMC) while the maltoside detergents DDM and Cymal 5 are effective at higher concentrations (8.7 CMC).

Fusion Construct Solubilization

The construction of fusion proteins, using glutathione S-transferase (GST), thioredoxin (TRX), or maltose binding protein (MBP) as the solubilizing fusion partners, has led to the expression of ANAT in a more soluble and active form (FIG. 2). While some solubilization was observed with these fusion constructs, the majority of each protein was still either associated with the membrane fraction or was found in inclusion bodies that are indicative of insoluble protein. However, ANAT activity was now measurable in the elution fractions from small scale Ni-IMAC purifications using either Trx-his-ANAT-his, GST-his-ANAT-his, or his-MBP-ANAT-his constructs. Unfortunately, further purification attempts using ion-exchange chromatography for the Trx-his-ANAT-his and GST-his-ANAT-his constructs, or additional affinity chromatography with a glutathione column for GST-his-ANAT-his or an amylose column for the his-MBP-ANAT-his construct, failed to significantly increase the purity of these ANAT enzyme forms.

The purity and subsequent yield of these ANAT fusion enzymes appears to be limited by two factors. First, the membrane anchor region still affects the inherent solubility of ANAT even in the presence of a solubilizing partner. More than 90% of the Trx-his-ANAT-his enzyme was still associated with the membrane fraction, and even with the more soluble his-MBP-ANAT-his construct more than half of the ANAT enzyme was still found to be membrane associated. Second, extensive protein cleavage was observed during E. coli cell growth and protein purification with each of the constructs. Attempts to use tandem affinity purification methodology to streamline the purification of these ANAT fusion proteins had only limited success. Regardless of which affinity step was applied first, either Ni-IMAC or a GST/amylose column, small bands corresponding to truncated ANAT fusion proteins were observed by SDS-PAGE as well as by Western blot analysis with anti-his tag antibodies. Similarly, truncations of the Trx-his-ANAT-his fusion protein were also observed by Western blot analysis with both the anti-His tag antibodies and an anti-Trx antibody. Further confirmation of the in-culture digestion of his-MBP-ANAT-his fusion protein was obtained by peptide mass fingerprinting analysis using MALDI-TOF mass spectrometry. The lower bands seen on SDS-PAGE and Western blotting after tandem affinity purification were shown to contain both a polyhis-tag and at least a portion of one of the affinity tag proteins (MBP or GST).

To completely separate the in-culture cleaved ANAT fusion proteins from the full length fusion enzyme, the amino-terminal polyhis tag of the his-MBP-ANAT-his construct was removed by site-directed mutagenesis. This allows separation of any truncated proteins since they would no longer contain a his tag, leading to an ideal construct for tandem affinity purification. The newly generated MBP-ANAT-his fusion enzyme was purified to more than 95% pure based on SDS-PAGE by a tandem affinity purification approach using Ni-NTA column as the first affinity step and an amylose column as the second affinity step. To increase the final yield of full length MBP-ANAT-his fusion enzyme, additional optimization of the cell growth conditions was performed. About 3 mg of purified MBP-ANAT-his fusion protein was obtained from 4 L of LB culture by decreasing the IPTG concentration for induction to 0.5 mM and by lowering the temperature after induction to 16° C.

The identification of full length ANAT from this MBP-ANAT-his construct was confirmed by peptide mass fingerprinting. For this verification seven peptides matched to the sequence of the human N-acetylaspartate synthetase (ANAT) were identified with high confidence (p<0.05) (FIGS. 8A-8C). The identification of the peptide IVAAEDHEALPGAK (SEQ ID NO: 4) near the N-terminus of ANAT and the peptide LFFQVR (SEQ ID NO: 5) near the C-terminus of the ANAT sequence supports the conclusion that the full length ANAT enzyme has been expressed and purified in a fusion construct with MBP.

Properties of Purified MBP-ANAT Fusion Enzyme

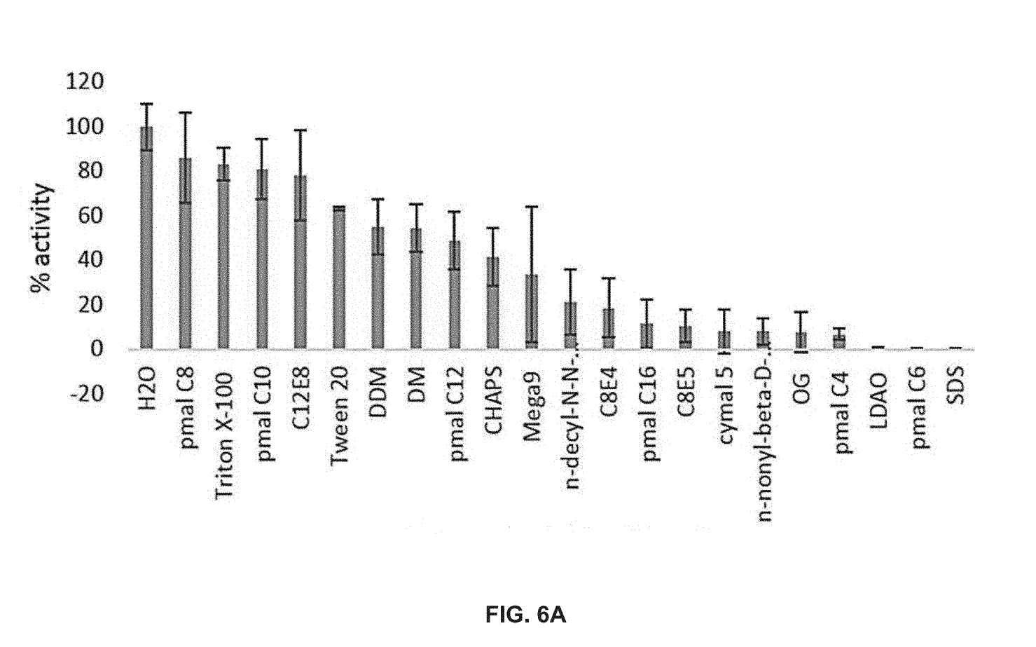

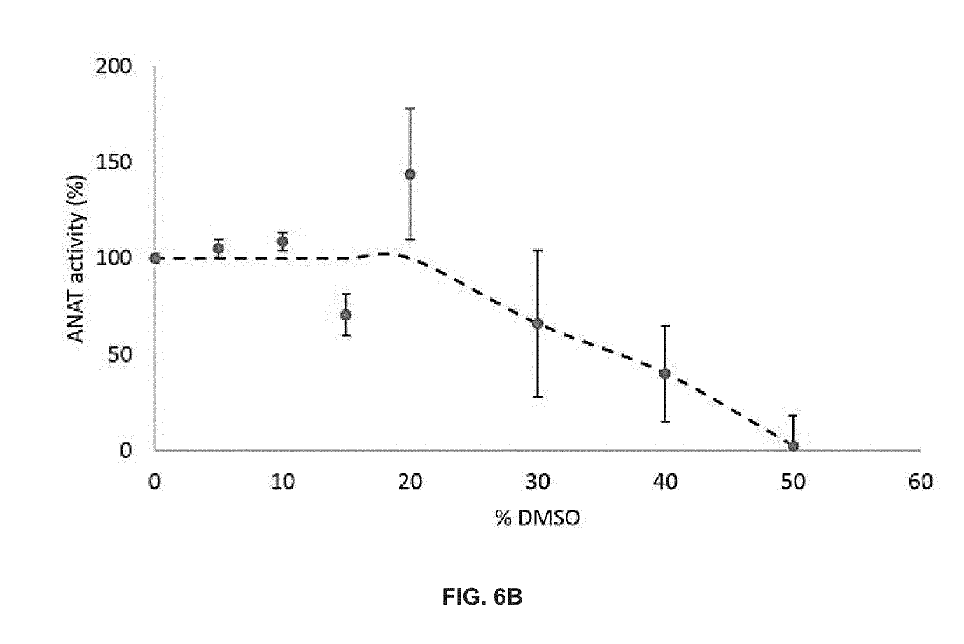

Now that the membrane-associated ANAT enzyme has been solubilized and purified, the properties of this enzyme were examined. The effect of different detergents on ANAT activity was evaluated by using the MBP-ANAT fusion protein. Non-ionic detergents such as Triton X-100 are generally found to be less disruptive to protein structures than ionic detergents such as SDS, and this same trend is seen for ANAT (FIG. 6A). In addition to the previously used Triton X-100, detergents such as C₁₂E₈, Tween 20, and several maltosides caused minimal disruption of ANAT, with greater than 50% residual activity after incubation with CMC levels of each of these detergents (FIG. 6A). In contrast, significant loss of activity was observed upon incubation with C₈ detergents, cymal5, octylglucoside, and some shorter chain polymaleic anhydride (pmal) detergents. An ionic detergent (SDS) and a zwitterionic detergent (LDAO) caused nearly complete loss of catalytic activity.

To evaluate the solubility of the MBP fusion enzyme, purified enzyme was concentrated up to 6 mg/ml and no precipitate or significant loss of activity was observed. By contrast, attempts to concentrate the native enzyme without the MBP fusion domain led to precipitation at protein concentrations less than 1 mg/ml. The MBP-ANAT fusion enzyme was stored at 4° C. for one week and the enzyme activity was assayed each day to evaluate the enzyme stability. No significant loss of specific activity was observed during storage under these conditions. The MBP-ANAT fusion enzyme was also subjected to multiple freeze-thaw cycles to identify long term storage conditions, with no appreciably decreased enzyme activity observed.

Kinetics of MBP-ANAT Fusion Enzyme

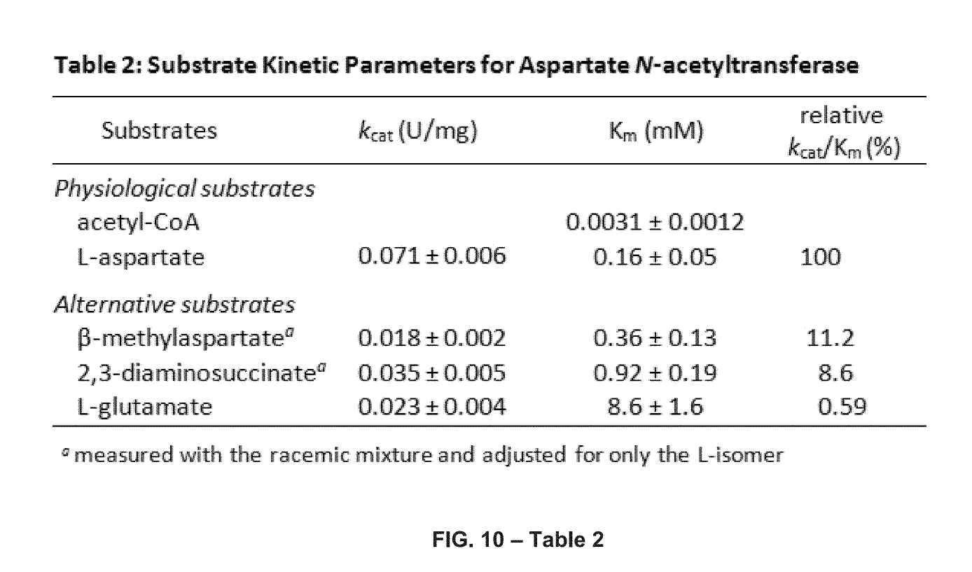

The kinetic parameters for the physiological substrates of ANAT were determined using the assay described above by varying the concentrations of both substrates and fitting the data to the equation for a sequential kinetic mechanism (eq. 1). The maximum velocity (Vₘₐₓ) of the MBP-ANAT fusion was determined to be 70 mU/mg of protein, with one activity unit defined as the production of one μmol of coenzyme A product per minute. The Kₘ of L-aspartate was determined to be 0.16 mM and the Kₘ of acetyl-CoA is 3.1 μM (Table 2, FIG. 10). A screen for alternative substrates was conducted using a customized amino acid library containing a total of 160 compounds. Of the compounds that were examined, only a few alternative substrates were identified. ANAT has less than 1% catalytic efficiency with L-glutamate, a one carbon longer homolog, and β-methylaspartate was identified as the best of these few alternative substrates (Table 2, FIG. 10).

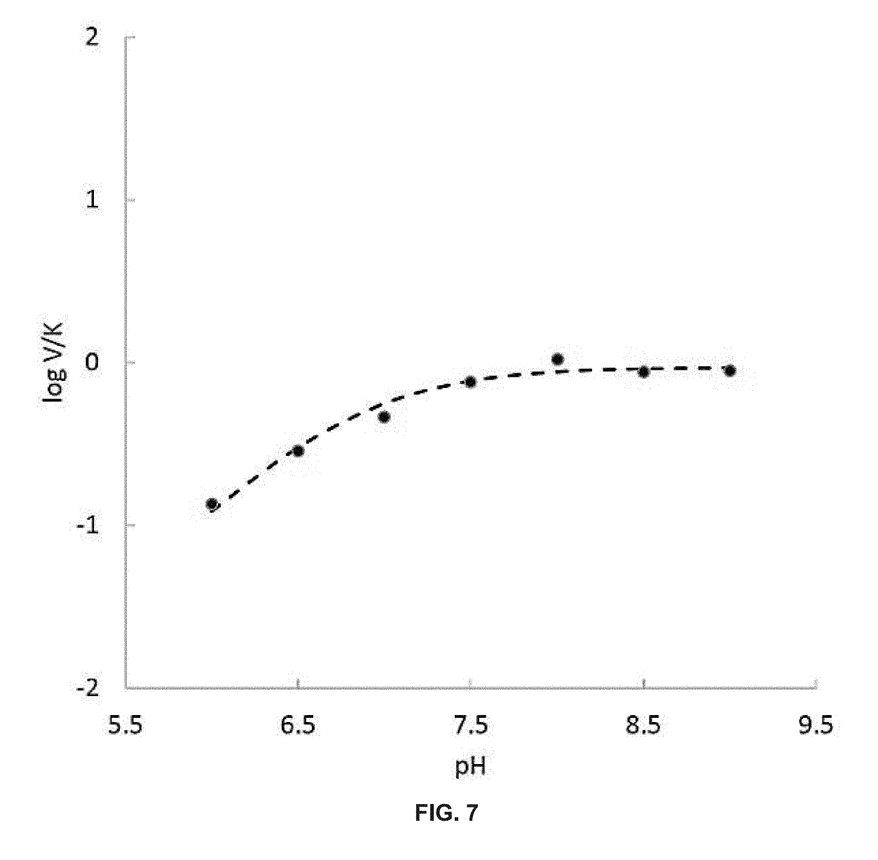

To measure the effects of pH on catalytic activity, and to characterize active site residues that are responsible for substrate binding and catalysis, pH profile studies were conducted. The enzymatic activity was observed to decrease at pH values below 7.5. A fit of the Vₘₐₓ/Kₘ data to a model that presumes the protonation of a single group leads to loss of activity (eq. 2) led to a pK value of 6.8±0.1 for a group that must be ionized for the enzyme to remain catalytically active (FIG. 7). The Vₘₐₓ profile did not show substantial changes across the pH range that was examined.

### DISCUSSION

Characterizing membrane-bound proteins remains a formidable challenge despite extensive studies with many different approaches that have been developed to lower the barriers. The initial challenge is to identify conditions that are sufficiently robust to extract the protein of interest from its lipid bilayer environment without irreversible denaturation. Once achieved, the next challenge is to determine conditions that will allow the protein to retain its native structure and its biological function in an aqueous environment that is dramatically different from its physiological state. These barriers are sufficiently high to severely limit the number of membrane proteins for which these aims have been achieved. These barriers have now been overcome for the extraction and stabilization of ANAT, a membrane-associated enzyme that plays an important role in the interrupted metabolism of NAA that is the underlying cause of Canavan disease.

Protein Solubilization

Overexpression of the nat8l gene that codes for ANAT leads to enhanced production of this enzyme, but ANAT remains associated with the cell membrane in an E. coli expression system. Under the standard growth and cell disruption conditions, there is a negligible level of ANAT observed in the soluble fractions, and the ANAT-his construct by itself does not enhance the level of soluble enzyme. Of the three different approaches that were examined to overcome this solubilization problem, the initial attempts at protein engineering of the putative membrane anchor region did not yield any active, soluble enzyme despite the elimination of most of this hydrophobic region. While this region does not appear to play any direct role in the catalytic function of ANAT, its removal likely leads to issues with protein folding that prevents the formation of a stable and active enzyme.

Detergent extraction of native ANAT was more successful, with several non-ionic detergents causing enhanced solubilization of the enzyme. However, the presence of these detergents complicated the affinity chromatography purification of ANAT, and the native enzyme is inherently unstable in an aqueous environment. Attempts to concentrate the enzyme led to precipitation, and there was also substantial loss of catalytic activity after storage for several days.

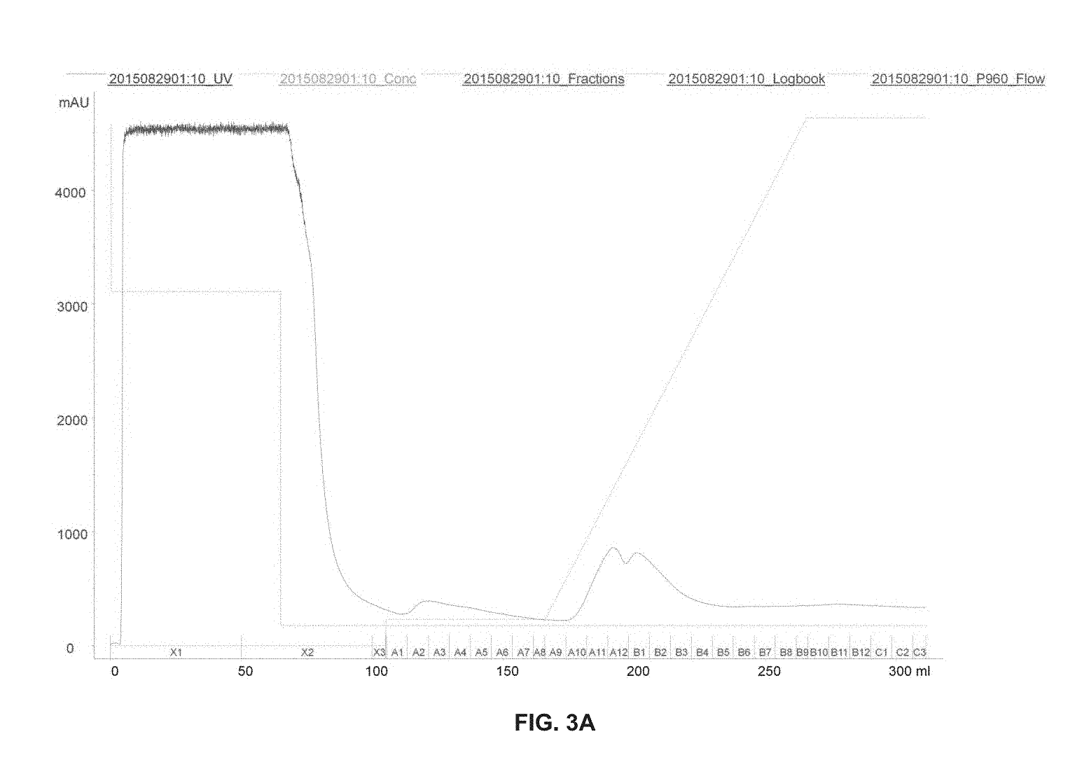

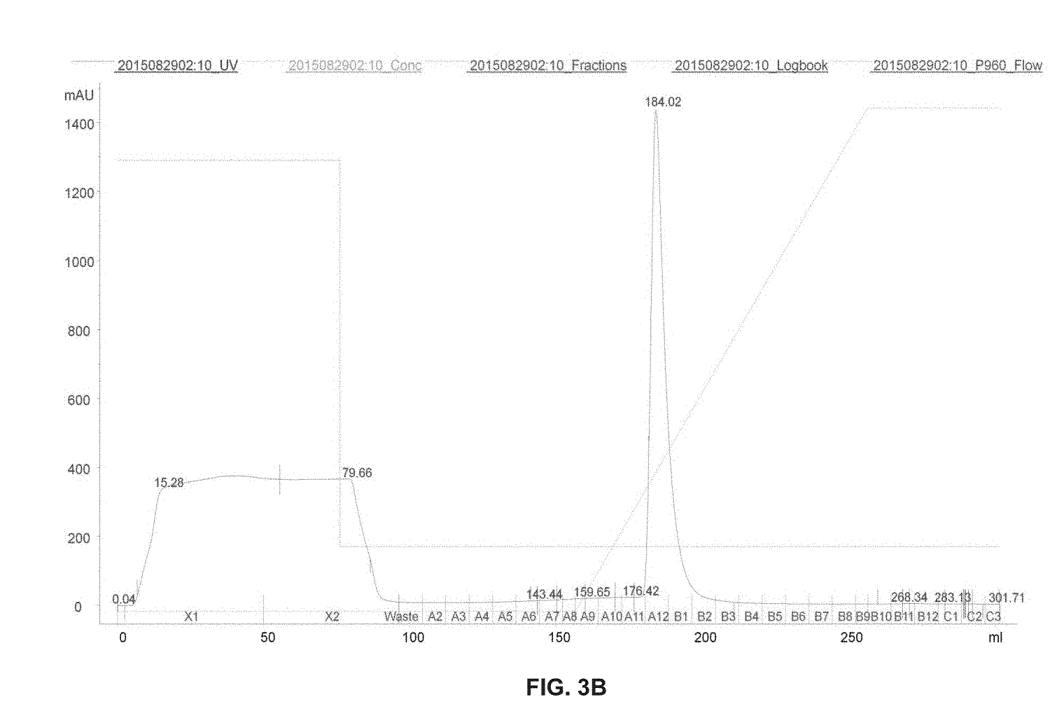

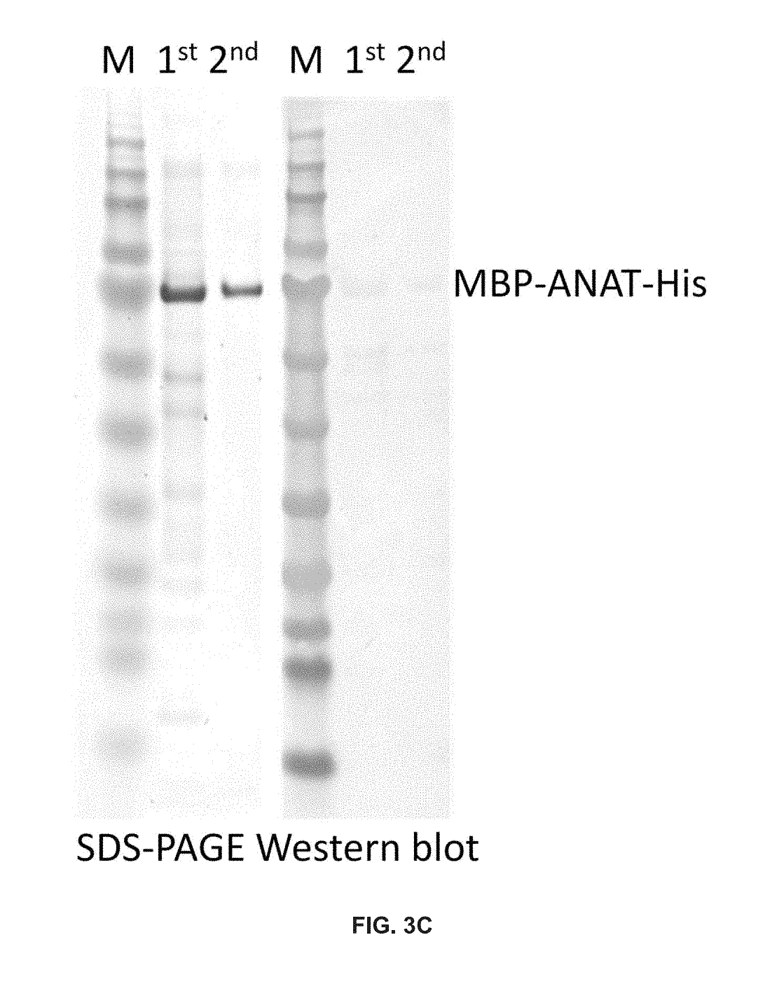

The most successful solubilization approach for ANAT was achieved through the incorporation of highly soluble fusion proteins to increase the overall hydrophilic surface of the fusion construct. Each of the fusion proteins tested led to some degree of soluble enzyme, with the MBP-fusion construct showing the highest solubility for ANAT. Subsequent tandem affinity purification, with Ni-IMAC followed by an amylose column, resulted in highly purified enzyme (FIGS. 3A-3C).

Protein Stability

The MBP-ANAT fusion enzyme is quite stable, with negligible loss of activity after storage for several days at 4° C. or through repeated freeze/thaw cycles. While the addition of a fusion domain such as MBP improves the solubility of ANAT, the presence of a hydrophobic membrane anchor region causes an increased tendency to aggregate at higher protein concentrations, leading to loss of activity. The addition of different detergents was examined in order to further increase the solubility of ANAT. In general, the non-ionic detergents that are less disruptive of protein structures had the least deleterious effects on enzyme activity, with ionic detergents having a significantly greater effect. Some subtle changes in detergent properties also led to profound differences in ANAT activity levels. Comparing the effects of poly maleic anhydride amphipols with different chain lengths found that less than 20% activity was lost by treatment with pmal-C8 and pmal-C10, while increasing the chain length to 12 to 16 carbons led to a progressive loss of >80% activity (Table 1, FIG. 9). On the other hand, decreasing the pmal detergent chain lengths to 4 to 6 carbons causes more than 90% loss of ANAT activity, indicating different modes of interactions with this structurally related family of detergents.

Enzyme Properties

Addition of the MBP fusion partner has improved the solubility of ANAT without having a significant effect on its catalytic activity. It is always difficult to quantitatively compare the properties of related enzymes that are obtained from different species, purified to different degrees, and assayed by different methods. However, the MBP-ANAT fusion has significantly higher catalytic turnover and lower substrate Kₘ values as compared to those parameters reported for the partially purified enzyme from rat brain, or the cell extracts of mouse ANAT that was expressed in HEK-293 cells. For comparison, the ANAT fusion enzyme with GST has similar kinetic parameters to the MBP fusion enzyme, with a Kₘ value of 0.1 mM for L-aspartate.

ANAT is also highly specific for L-aspartate as the acetyl group acceptor. Subtle changes in the amino acid structure of the substrate, such as the introduction of a methyl group (β-methylaspartate), or extending the structure by one methylene group (L-glutamate), leads to a profound loss of activity (Table 2, FIG. 10). Even the introduction of a second amino group on the same carbon skeleton (2,3-diaminosuccinate) gave an alternative substrate with less than 10% the efficiency of the physiological substrate. All of the other amino acids tested, a total of 160, did not function as substrates for this enzyme.

The application of pH profile studies can identify functional groups that must be in the correct ionization state to participate in the enzyme-catalyzed reaction. The Vₘₐₓ profile, indicative of changes in ionization in the enzyme-substrate complex, did not show substantial changes in the pH range from 6 to 9.5. However, there is an enzyme group identified in the Vₘₐₓ/Kₘ profile, with a pK value of 6.8 (consistent with the imidazole group of a histidine), that must be ionized for the enzyme to be active (FIG. 7). Sequence alignment of five related ANATs from different species shows that only a single histidine, His-256, located in a homologous region in the putative catalytic domains of ANAT, is fully conserved, while five other histidines are found to be conserved in four of these enzymes.

Aspartate N-acetyltransferase has been found to play a central role in the disrupted metabolism of NAA, the underlying cause of Canavan disease. Solubilization of the membrane-associated enzyme has been successfully achieved through the construction of fusion proteins with solubilizing partners. As one example, the MBP-ANAT fusion enzyme has been extracted and purified, and its stability and kinetic properties have been evaluated, allowing for the development of inhibitors for the treatment of Canavan disease.

Certain embodiments of the fusion proteins, amino acid constructs, and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A fusion protein comprising an amino acid construct between an N-terminus and a C-terminus, the amino acid construct comprising N-acetylaspartate synthetase (ANAT) [SEQ ID NO: 1] and at least one solubilizing partner; the amino acid construct optionally comprising one or more polyhistidine (his) tags.

2. The fusion protein of claim 1, wherein the solubilizing partner is selected from the group consisting of glutathione S-transferase (GST), thioredoxin (TRX), and maltose binding protein (MBP).

3. (canceled)

4. (canceled)

5. (canceled)

6. (canceled)

7. The fusion protein of claim 1, comprising a MBP-ANAT sequence.

8. (canceled)

9. The fusion protein of claim 1, comprising a TRX-his-ANAT-his sequence.

10. (canceled)

11. The fusion protein of claim 1, comprising a GST-his-ANAT-his sequence.

12. (canceled)

13. The fusion protein of claim 1, comprising a his-MBP-ANAT-his sequence.

14. (canceled)

15. (canceled)

16. The fusion protein of claim 1, wherein the fusion protein is solubilized in a detergent.

17. (canceled)

18. The fusion protein of claim 16, wherein the detergent is selected from the group consisting of Triton X-100, SDS, C₁₂E₈, Tween 20, DDM, and Cymal 5.

19. (canceled)

20. (canceled)

21. (canceled)

22. (canceled)

23. A variant fusion protein comprising an amino acid sequence having at least about 75%, 85%, 90% 0r 95% sequence identity to the fusion protein of claim 1.

24. (canceled)

25. (canceled)

26. (canceled)

27. A method of constructing a fusion protein, the method comprising:
constructing a polyhistidine-tagged fusion protein having an amino acid construct containing ANAT and at least one solubilizing partner; and
exposing the polyhistidine-tagged fusion protein to a mutagen to produce a fusion protein.

28. The method of claim 27, wherein the solubilizing partner is selected from the group consisting of GST, TRX, and MBP.

29. (canceled)

30. (canceled)

31. A method of purifying N-acetylaspartate synthetase (ANAT), the method comprising:
constructing the fusion protein of claim 1 by fusing ANAT with at least one solubilizing partner; and
subjecting the fusion protein to Ni-immobilized metal affinity chromatography to purify the fusion protein.

32. The method of claim 31, wherein the at least one solubilizing partner is selected from the group consisting of glutathione S-transferase (GST), thioredoxin (TRX), and maltose binding protein (MBP).

33. The method of claim 31, wherein the Ni-immobilized metal affinity chromatography comprises a tandem affinity process involving a Ni-NTA column and a second chromatography step.

34. (canceled)

35. A method of solubilizing N-acetylaspartate synthetase (ANAT), the method comprising:
constructing the fusion protein of claim 1 by fusing ANAT with at least one solubilizing partner; and
incubating the fusion protein in a detergent at or above the critical micelle concentration to solubilize the fusion protein.

36. The method of claim 35, wherein the solubilizing partner is selected from the group consisting of glutathione S-transferase (GST), thioredoxin (TRX), and maltose binding protein (MBP).

37. (canceled)

38. (canceled)

39. A method of developing a treatment for Canavan disease, the method comprising:
fusing ANAT with at least one solubilizing partner to create a soluble ANAT fusion protein of claim 1; and
testing inhibitor candidates against the soluble ANAT fusion protein to develop a treatment for Canavan disease.

40. The method of claim 39, wherein the at least one solubilizing partner is selected from the group consisting of glutathione S-transferase (GST), thioredoxin (TRX), and maltose binding protein (MBP).

41. A method for making a fusion protein, the method comprising:
cloning a human nat8l gene into a plasmid, wherein the plasmid contains a gene for at least one solubilizing partner;
transforming E. coli cells with the plasmid;
growing the E. coli cells for a period of time; and
inducing protein expression in the E. coli cells to produce a fusion protein.

42. The method of claim 41, wherein the at least one solubilizing partner is selected from the group consisting of thioredoxin (TRX), glutathione S-transferase (GST), or maltose binding protein (MBP)

43. (canceled)

44. (canceled)
